(19) United States
(12) Patent Application Publication
(10) Pub. No.: US 2025/0368597 A1
(43) Pub. Date: Dec. 4, 2025

(54) PROCESS FOR THE INTRACYCLIC DOUBLE BOND ISOMERIZATION

(71) Applicant: Firmenich SA, Satigny (CH)

(72) Inventors: Denis JACOBY, Satigny (CH); Jean-Jacques RIEDHAUSER, Satigny (CH); Luigi MARINONI, Satigny (CH); Xavier MAILLARD, Satigny (CH); Mickaël HENRION, Satigny (CH)

(21) Appl. No.: 18/869,188

(22) PCT Filed: Jun. 7, 2023
(86) PCT No.: PCT/EP23/65220

§ 371 Date: Nov. 25, 2024

(30) Foreign Application Priority Data
Jun. 9, 2022 (EP) 22177997.8

(51) Int. Cl. C07C 45/67

(52) U.S. Cl. CPC C07C 45/67

(57) ABSTRACT

Disclosed herein is a process for preparing a 1-(cycloalk-1-en-1-yl) acyl or 1-(cycloalkadien-1-yl) acyl derivative including isomerizing a conjugated intracyclic carbon-carbon double bond in the presence of a catalyst system including palladium (Pd) and molecular hydrogen or a hydrogen source.

### TECHNICAL FIELD

The present invention relates to the field of organic synthesis. More particularly, it provides a process for the preparation of a 1-(cycloalk-1-en-1-yl) acyl or 1-(cycloalkadien-1-yl) acyl derivative comprising a conjugated intracyclic carbon-carbon double bond isomerization in the presence of a catalyst system comprising palladium (Pd) and molecular hydrogen or a hydrogen source.

### BACKGROUND

Many 1-(cycloalk-1-en-1-yl) acyl or 1-(cycloalkadien-1-yl) acyl derivative as defined in formula (I) are useful products as such or useful intermediates for the preparation of other important raw materials. The compounds of formula (I) are of particular interest to the perfumery industry, and in particular 1-(3,3-dimethylcyclohex-1-en-1-yl)ethan-1-one being an important intermediate for the preparation of industrially relevant compounds such as Dynascone® (1-(5,5-dimethyl-1-cyclohexen-1-yl)-4-penten-1-one; origin: Firmenich SA). Most of the preparations of 1-(cycloalk-1-en-1-yl) acyl 1-(cycloalkadien-1-yl) acyl derivative lead to the undesired regioisomers or a mixture of regioisomers wherein the double bond(s) are not at the correct position requiring a supplementary isomerization reaction. However, the isomerization reaction of a conjugated intracyclic carbon-carbon double bond to obtain a 1-(cycloalk-1-en-1-yl) acyl or 1-(cycloalkadien-1-yl) acyl derivative has never been reported. So, there is a need to develop such isomerization.

The present invention allows obtaining 1-(cycloalk-1-en-1-yl) acyl or 1-(cycloalkadien-1-yl) acyl derivative via an isomerization of intramolecular double bound(s) with high selectivity in the presence of a catalyst system comprising palladium (Pd) and molecular hydrogen or a hydrogen source.

### DESCRIPTION OF THE INVENTION

We have now found that the 1-(cycloalk-1-en-1-yl) acyl or 1-(cycloalkadien-1-yl) acyl derivative can be produced in an advantageous manner by means of a catalytic isomerization as described and result in minimal by product formation and high productivity.

Therefore, a first object of the present invention is a process for the preparation of a 1-(cycloalk-1-en-1-yl) acyl or 1-(cycloalkadien-1-yl) acyl derivative comprising the isomerization of intracyclic carbon-carbon double bond wherein said isomerization is carried out by contacting a substrate comprising a conjugated intracyclic carbon-carbon double bond with a catalyst system comprising
i) palladium (Pd); and
ii) molecular hydrogen or a hydrogen source.

For the sake of clarity, it is understood that by the expression “hydrogen source” it is intended the usual meaning in the art, i.e. a compound capable of producing molecular hydrogen (i.e. H₂), hydrogen atom or the equivalent in the reaction medium.

For the sake of clarity, it is understood that by the expression “a conjugated intracyclic carbon-carbon double bond” it is intended the usual meaning in the art, i.e. the substrate comprises at least one double bond which is conjugated with the acyl functional group.

According to any embodiments of the invention, the 1-(cycloalk-1-en-1-yl) acyl or 1-(cycloalkadien-1-yl) acyl derivative is a compound of formula

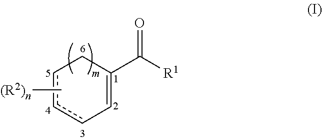

in the form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together, form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

According to any embodiments of the invention, the substrate is a compound of formula

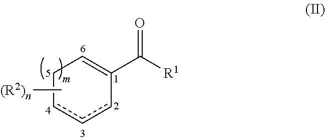

in the form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

According to any embodiments of the invention, the 1-(cycloalk-1-en-1-yl) acyl or 1-(cycloalkadien-1-yl) acyl derivative is a compound of formula

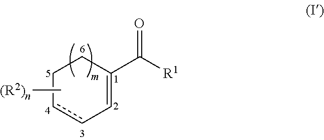

in the form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together, form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

According to any embodiments of the invention, the substrate is a compound of formula

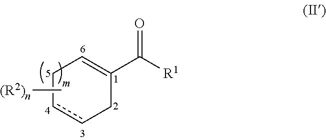

in the form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

For the sake of clarity, by the expression “any one of its stereoisomers or a mixture thereof”, or the similar, it is meant the normal meaning understood by a person skilled in the art, i.e. that the compound of formula (I) or compound of formula (II) can be a pure enantiomer or diastereomer. In other words, the compound of formula (I) or compound of formula (II) may possess several stereocenters and each of said stereocenter can have two different stereochemistries (e.g. R or S). The compound of formula (I) or compound of formula (II) may even be in the form of a pure enantiomer or in the form of a mixture of enantiomers or diastereoisomers. The compound of formula (I) or compound of formula (II) can be in a racemic form or scalemic form. Therefore, the compound of formula (I) or compound of formula (II) can be one stereoisomer or in the form of a composition of matter comprising, or consisting of, various stereoisomers.

For the sake of clarity, by the expression “one dotted line is a carbon-carbon single bond and the other is a carbon-carbon single bond or a carbon-carbon double bond”, or the similar, it is meant the normal meaning understood by a person skilled in the art, i.e. that the whole bonding (solid and dotted line) between the carbon atoms connected by said dotted line is a carbon-carbon single or double bond. In other words, the compound of formula (I) and (II) are a cycloalkenyl or a cycloalkadienyl derivative. The person skilled in the art is well aware that, when one dotted line is a carbon-carbon double bond, then the adjacent dotted line cannot be a double bond. In other words, compound of formula (I) may be a 1-(cycloalken-1-yl) acyl of formula (Ia), or 1-(cycloalkadien-1-yl) acyl of formula (Ib)

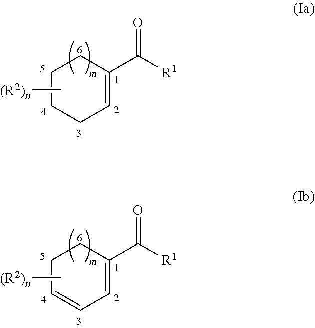

in the form of any one of its stereoisomers or as a mixture thereof, wherein m, n, R¹ and R² have the same meaning as defined above
and
compound of formula (II) may be of formula (IIa) or (IIb)

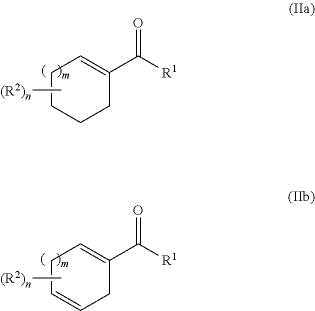

in the form of any one of its stereoisomers or as a mixture thereof, wherein m, n, R¹ and R² have the same meaning as defined above.

The terms “alkyl”, “alkoxyl” and “alkenyl” are understood as comprising branched and linear alkyl, alkoxyl and alkenyl groups. The terms “alkenyl” or “cycloalkenyl” are understood as comprising 1 olefinic double bond. The terms “cycloalkyl” or “cycloalkenyl” are understood as comprising a monocyclic group.

For the sake of clarity, by the expression “R² represents substituents of the ring”, or the similar, it is meant the normal meaning understood by a person skilled in the art, i.e. that said group is bound to the ring at any one of the available positions.

The term “optionally” is understood that a certain group to be optionally substituted can or cannot be substituted with a certain functional group. The term “one or more” is understood as being substituted with 1 to 7, preferably 1 to 5, preferably 1 to 3 and more preferably 1 to 2 of a certain functional group.

For the sake of clarity, by the expression “two R² groups or R¹ and one R² group, taken together, form a C₅₋₈ cycloalkyl or C₅₋₈ cycloalkenyl group”, it is meant that the carbon atom to which both groups are bonded is included into the C₅₋₈ cycloalkyl or C₅₋₈ cycloalkenyl group.

According to any embodiments of the invention, when the substrate is a compound of formula (IIb), the isomerization leads to the preparation of 1-(cycloalkadien-1-yl) acyl derivative of formula (Ib) or to a composition of matter comprising at least 50% of 1-(cycloalkadien-1-yl) acyl derivative of formula (Ib) and at most 50% of 1-(cycloalkadien-1-yl) acyl derivative of formula (A)

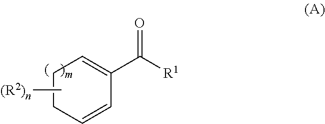

in the form of any one of its stereoisomers or as a mixture thereof, wherein m, n, R¹ and R² have the same meaning as defined above.

According to any embodiments of the invention, the dotted lines are single bond.

According to any embodiments of the invention, the 1-(cycloalk-1-en-1-yl) acyl derivative is a compound of formula

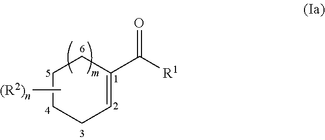

in the form of any one of its stereoisomers or a mixture thereof and wherein m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together, form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

According to any embodiments of the invention, the substrate is a compound of formula

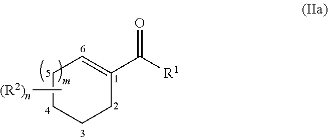

in the form of any one of its stereoisomers or a mixture thereof and wherein m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

According to any embodiments of the invention, the compound of formula (II) and the compound of (I) are different; i.e. the R² groups are such that the compound of formula (II) is not the compound of formula (I), particularly the compound of formula (II′) and the compound of (I′) are different; i.e. the R² groups are such that the compound of formula (IIa) is not the compound of formula (Ia), even more particularly the compound of formula (IIa) and the compound of (Ia) are different; i.e. the R² groups are such that the compound of formula (IIa) is not the compound of formula (Ia).

According to any embodiments of the invention, the compound of formula (II), (II′) or (IIa) and the compound of formula (I), (I′) or (Ia) may comprise at least one R² group in position 2, 3, 5 or 6 or in position 2, 3, 4 or 5. Particularly, the compound of formula (II), (II′) or (IIa) and the compound of formula (I), (I′) or (Ia) may comprise at least one R² group in position 3 or 5 or in position 4 or 5. Particularly, the compound of formula (II), (II′) or (IIa) and the compound of formula (I), (I′) or (Ia) may comprise at least one R² group in position 5. Even more particularly, the compound of formula (II), (II′) or (IIa) and the compound of formula (I), (I′) or (Ia) may comprise at least two R² group in position 5.

According to any embodiments of the invention, m may be an integer comprised between 0 and 5. Particularly, m may be an integer comprised between 0 and 3. Particularly, m may be an integer between 0 and 2. Particularly, m may be 0 or 1. Even more particularly, m may be 1.

According to any embodiments of the invention, R², simultaneously or independently, may be a C₁₋₄ alkyl or C₂₋₄ alkenyl group; or two R² group, taken together form a C₅₋₆ cycloalkyl group optionally substituted by one or more C₁₋₄ alkyl, C₂₋₄ alkenyl or C₁₋₄ alkoxy group. Particularly, R², simultaneously or independently, may be a C₁₋₃ alkyl or C₂₋₄ alkenyl group; or two R² group, taken together form a C₅₋₆ cycloalkyl group substituted by one or more C₁₋₃ alkyl, C₂₋₃ alkenyl or C₁₋₃ alkoxy group. Particularly, R², simultaneously or independently, may be a C₁₋₃ alkyl or C₂₋₄ alkenyl group; or two R² group, taken together form a C₅₋₆ cycloalkyl group substituted by one or more C₁₋₂ alkyl C₁₋₂ alkoxy group. Particularly, R², simultaneously or independently, may be a C₁₋₃ alkyl group; or two R² group, taken together form a C₅₋₆ cycloalkyl group. Particularly, R², simultaneously or independently, may be a methyl or an ethyl group. More particularly, R² may be a methyl group.

According to any embodiments of the invention, n may be an integer comprised between 1 and 6, even between 1 and 4, even between 1 and 3, even more between 1 and 2. Particularly, n may be 1 or 2 or 3. Even more particularly, n may be 2.

According to any embodiments of the invention, the compound of formula (I) is a compound of formula

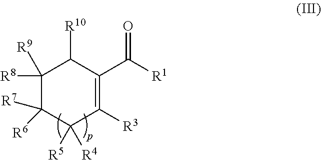

in the form of any one of its stereoisomers or as a mixture thereof, wherein p is 0 or 1; R¹ is a C₁₋₆ alkyl, C₂₋₆ alkenyl or C₁₋₆ alkoxy group; each R³, R⁴, R⁵, R⁶, R⁷, R⁸, R⁹ and R¹⁰, independently from each other, represent a hydrogen atom, a C₁₋₆ alkyl or C₂₋₆ alkenyl group; or two groups among R³, R⁴, R⁵, R⁶, R⁷, R⁸, R⁹ and R¹⁰ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R³ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R⁴ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; provided that R³, R⁴, R⁵, R⁶, R⁷, R⁸, R⁹ and R¹⁰ are such that compound of formula (III) is different from compound of formula (IV) as defined below.
   and the compound of formula (II) is of formula

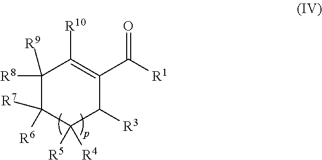

in the form of any one of its stereoisomers or as a mixture thereof, wherein p, R¹, R³, R⁴, R⁵, R⁶, R⁷, R⁸, R⁹ and R¹⁰ have the same meaning as defined above.

According to any embodiments of the invention, the compound of formula (III) is not identical to compound of formula (IV). In other words, the compound of formula (III) is different to compound of formula (IV)

According to any embodiments of the invention, p is 1. In other words, the compound of formula (III) is a compound of formula

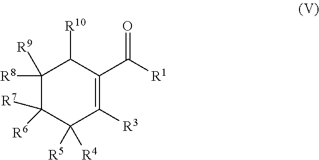

in the form of any one of its stereoisomers or as a mixture thereof, wherein R¹ is a C₁₋₆ alkyl, C₂₋₆ alkenyl or C₁₋₆ alkoxy group; each R³, R⁴, R⁵, R⁶, R⁷, R⁸, R⁹ and R¹⁰, independently from each other, represent a hydrogen atom, a C₁₋₆ alkyl or C₂₋₆ alkenyl group; or two groups among R³, R⁴, R⁵, R⁶, R⁷, R⁸, R⁹ and R¹⁰ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R³ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R⁴ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; provided that R³, R⁴, R⁵, R⁶, R⁷, R⁸, R⁹ and R¹⁰ are such that compound of formula (V) is different from compound of formula (VI) as defined below.
   and the compound of formula (IV) is of formula

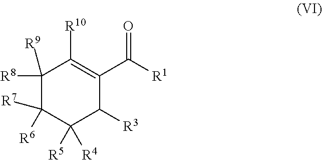

in the form of any one of its stereoisomers or as a mixture thereof, wherein R¹, R³, R⁴, R⁵, R⁶, R⁷, R⁸, R⁹ and R¹⁰ have the same meaning as defined above.

According to any embodiments of the invention, the compound of formula (V) is not identical to compound of formula (VI). In other words, the compound of formula (V) is different to compound of formula (VI)

According to any embodiments of the invention, at least one group among R⁴, R³; R¹⁰ and R⁹ is not a hydrogen atom and compound of formula (III) or (V) wherein R³ is identical to R¹⁰, R⁴ is identical to R⁹, R⁵ is identical to R⁸, is excluded. Particularly, at least one group among R⁴ and R⁹ is not a hydrogen atom.

According to any embodiments of the invention, R³, R⁴, R⁵, R⁷ and R¹⁰, independently from each other, may represent a hydrogen atom, a C₁₋₄ alkyl or C₂₋₄ alkenyl group. Particularly, R³, R⁴, R⁵, R⁷ and R¹⁰, independently from each other, may represent a hydrogen atom or a C₁₋₃ alkyl group. Particularly, R³, R⁴, R⁵, R⁷ and R¹⁰, independently from each other, may represent a hydrogen atom or a C₁₋₂ alkyl group. Even more particularly, R³, R⁴, R⁵, R⁷ and R¹⁰ may represent a hydrogen atom.

According to any embodiments of the invention, the compound of formula (V) is a compound of formula

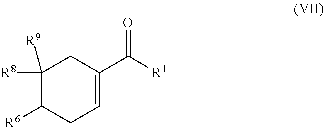

in the form of any one of its stereoisomers or as a mixture thereof, wherein R¹ is a C₁₋₆ alkyl, C₂₋₆ alkenyl or C₁₋₆ alkoxy group; each R⁶, R⁸ and R⁹, independently from each other, represent a hydrogen atom, a C₁₋₆ alkyl or C₂₋₆ alkenyl group; or R⁸ and R⁹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group;
   and the compound of formula (VI) is of formula

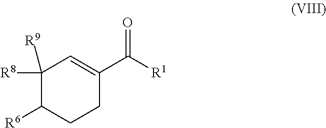

in the form of any one of its stereoisomers or as a mixture thereof, wherein R¹, R⁶, R⁸ and R⁹ have the same meaning as defined above.

According to any embodiments of the invention, at least one group among R⁹, R⁸; and R⁶ is not a hydrogen atom.

According to any embodiments of the invention, R⁶ may represent a hydrogen atom, a C₁₋₄ alkyl or C₂₋₄ alkenyl group. Particularly, R⁶ may represent a hydrogen atom or a C₁₋₃ alkyl group. Particularly, R⁶ may represent a hydrogen atom or a C₁₋₂ alkyl group. Particularly, R⁶ may represent a hydrogen atom or a methyl group. Even more particularly, R⁶ may represent a hydrogen atom.

According to any embodiments of the invention, the compound of formula (VII) is not identical to compound of formula (VIII).

According to any embodiments of the invention, the compound of formula (VII) is a compound of formula

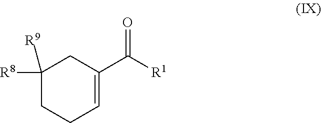

in the form of any one of its stereoisomers or as a mixture thereof, wherein R¹ is a C₁₋₆ alkyl, C₂₋₆ alkenyl or C₁₋₆ alkoxy group; each R⁸ and R⁹, independently from each other, represent a hydrogen atom, a C₁₋₆ alkyl or C₂₋₆ alkenyl group; or R⁸ and R⁹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group;
   and the compound of formula (VIII) is of formula

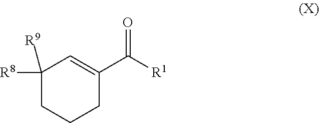

in the form of any one of its stereoisomers or as a mixture thereof, wherein R¹, R⁸ and R⁹ have the same meaning as defined above.

According to any embodiments of the invention, at least one group among R⁹ and R⁸ is not a hydrogen atom.

According to any embodiments of the invention, R⁸ may represent a hydrogen atom, a C₁₋₄ alkyl or C₂₋₄ alkenyl group. Particularly, R⁸ may represent a hydrogen atom, a C₁₋₃ alkyl or C₂₋₃ alkenyl group. Particularly, R⁸ may represent a hydrogen atom or a C₁₋₂ alkyl group. Even more particularly, R⁸ may represent a methyl group.

According to any embodiments of the invention, R⁹ may represent a hydrogen atom, a C₁₋₄ alkyl or C₂₋₄ alkenyl group. Particularly, R⁹ may represent a hydrogen atom, a C₁₋₃ alkyl or C₂₋₃ alkenyl group. Particularly, R⁹ may represent a hydrogen atom or a C₁₋₂ alkyl group. Even more particularly, R⁹ may represent a methyl group.

According to any embodiments of the invention, R¹ may be a C₁₋₄ alkyl, C₂₋₄ alkenyl or C₁₋₄ alkoxy group. Particularly, R¹ may be a C₁₋₃ alkyl, C₂₋₃ alkenyl or C₁₋₃ alkoxy group. Particularly, R¹ may be a C₁₋₃ alkyl group. Particularly, R¹ may be a methyl, ethyl or propyl group. Even more particularly, R¹ may be a methyl group.

Non-limiting examples of suitable compounds of formula (I) may include 1-(5,5-dimethylcyclohex-1-en-1-yl)ethan-1-one, 1-(5-ethyl-5-methylcyclohex-1-en-1-yl)ethan-1-one, 1-(5,5-dimethylcyclohexa-1,3-dien-1-yl)ethan-1-one, 1-(4,5,5-trimethylcyclohex-1-en-1-yl)ethan-1-one, 1-(4-isopropyl-4-methylcyclopent-1-en-1-yl)ethan-1-one.

Non-limiting examples of suitable compounds of formula (II) may include 1-(3,3-dimethylcyclohex-1-en-1-yl)ethan-1-one, 1-(3-ethyl-3-methylcyclohex-1-en-1-yl)ethan-1-one, 1-(3,3-dimethylcyclohexa-1,4-dien-1-yl)ethan-1-one, 1-(3,3,4-trimethylcyclohex-1-en-1-yl)ethan-1-one, 1-(3-isopropyl-3-methylcyclopent-1-en-1-yl)ethan-1-one.

According to any embodiments of the invention, the catalyst system comprises palladium (Pd) in a form of a homogeneous complex or in elemental metallic form. Particularly, the catalyst system comprises palladium (Pd) in elemental metallic form. Suitable forms of such metal for carrying out chemical reactions are well known to a person skilled in the art.

According to any one of the above embodiments of the invention, said palladium (Pd) is supported on a carrying material.

For the sake of clarity, by carrying material it is intended a material wherein it is possible to deposit such metal and which is inert toward the hydrogen source and the substrate.

According to any one of the above embodiments of the invention, specific and non-limiting examples of carrying material is carbon, silica or aluminum oxide. Such supports are well known to a person skilled in the art.

The supported palladium (Pd) are known compounds and are commercially available. A person skilled in the art is able to select the preferred kind of metal as the way that it was deposit on the support, as the proportion of metal on support material, as the form (powder, granules, pellets, extrudates, mousses . . . ) and as the surface area of the support.

According to any one of the above embodiments of the invention, the amount of metal relative to the support can range between 0.05% and 25% w/w, or even between 1% and 6%, relative to the weight on the support used.

The palladium (Pd), in a supported form or as such, can be added into the reaction medium of the invention's process in a large range of concentrations. As non-limiting examples, one can cite as metal concentration values those ranging from 0.01 mol % to 10 mol %, relative to the total amount of substrate. Preferably, the metal concentration will be comprised between 0.02 mol % to 5 mol %, or even between 0.04 mol % to 2 mol %. It goes without saying that the optimum concentration of metal will depend, as the person skilled in the art knows, on the nature of the latter, on the nature of the substrate, if the process is run in batch or continuously, on the temperature and on the pressure of H₂ used during the process, as well as the desired time of reaction.

The supported palladium may be recycled at the end of the invention's process. In other words, the supported palladium may be recovered at the end of the invention's process and use several times in the invention's process.

The process according to the invention is carried out in the presence of molecular hydrogen or hydrogen source.

According to any one of the above embodiments of the invention, said hydrogen source can be a transfer hydrogenation agent. Specific and non-limiting examples of catalytic transfer hydrogenation agents are tetralin, formic acid, formate salt (such as sodium formate, potassium formate or ammonium formate), limonene or a mixture thereof. Particularly, the transfer hydrogenation agent may be tetralin, formic acid, formate salt, limonene or a mixture thereof. Even more particularly, the transfer hydrogenation agent may be formic acid, formate salt, limonene or a mixture thereof.

The transfer hydrogen agent can be added into the reaction medium of the invention's process in a large range of concentrations. As non-limiting examples, one can cite as hydrogen source concentration values those ranging from 0.01 mol % to 100 mol %, or even between 0.01 mol % to 10 mol %, or even more between 0.01 mol % to 5 mol % relative to the amount of the substrate. A large amount of transfer hydrogenation agent is used when only a small part generates molecular hydrogen. For instance, approximately around 10% of tetralin are converted into molecular hydrogen. It goes without saying that the optimum concentration of hydrogen source will depend, as the person skilled in the art knows, on the nature of the latter, on the nature of the substrate, of the temperature and on the catalyst used during the process, as well as the desired time of reaction.

According to any one of the above embodiments of the invention, as an alternative to the transfer hydrogenation agent, the molecular hydrogen can be used pure or mixed with an inert gas. Specific and non-limiting examples of such inert gas are nitrogen or argon. The H₂/inert gas volume ratio is comprised between 1/1 to 0.01/1 and more preferably the ratio is 0.05/1.

The molecular hydrogen can be added into the reaction medium of the invention's process in a large range of concentrations. As non-limiting examples, one can cite as molecular hydrogen concentration values those ranging from 0.01 mol % to 100 mol %, relative to the amount of the substrate. Preferably, the hydrogen source concentration will be comprised between 0.01 mol % to 10 mol % relative to the amount of the substrate. Preferably, the hydrogen source concentration will be comprised between 0.01 mol % to 8 mol % relative to the amount of the substrate. Even more preferably, the hydrogen source concentration will be comprised between 0.01 mol % to 5 mol % relative to the amount of the substrate. Of course, a person skilled in the art is well able to adjust the pressure or the flow (e.g. in a continuous process) of molecular hydrogen to obtain this range of concentration as a function of the process is batch or continuous. The person skilled in the art is also well able to adjust the concentration of molecular hydrogen as a function of the catalyst load and of dilution of the substrate in the solvent.

From 0.01 mol % to 10 mol %, even from 0.01 mol % to 8 mol %, even more from 0.01 mol % to 5 mol % of the hydrogen source or the molecular hydrogen, relative to the amount of the substrate, is present in the invention's process.

The invention's process can be carried out under batch or continuous conditions. According to a particular embodiment of the invention, the process is a continuous one, as it allows higher productivity.

The reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, 1,3-diisopropylbenzene, paracymene, cumene, pseudocumene, benzyl acetate, xylene or a mixture thereof, C₃₋₁₆ alkane such as hexadecane, ether solvents such as tetrahydrofuran, butyl ether, methyltetrahydrofuran or a mixture thereof. Particularly, the solvent has a boiling point above 100° C. The choice of the solvent is a function of the nature of the substrate and of the complex and the person skilled in the art is well able to select the solvent most convenient in each case to optimize the reaction.

The temperature at which the isomerization can be carried out is comprised between 120° C. and 300° C. More preferably in the range of between 150° C. and 250° C. for a continuous process and between 150° C. to 200° C. for a batch process. Of course, a person skilled in the art is also able to select the preferred temperature as a function of the melting and boiling point of the starting and final products as well as the desired time of reaction or conversion.

According to any one of the above embodiments of the invention, the compound of formula (II) may be prepared according to several methods known in the art such as Diels-Alder, cyclisation or Friedel Craft reaction. The person skilled in the art will be able to select best conditions to prepare compound of formula (II). Particularly, the invention's process further comprises the preparation of the compound of formula (IIa) from compound of formula

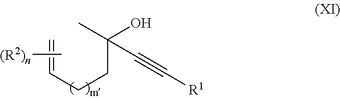

in the form of any one of its stereoisomers or a mixture thereof; and wherein n, R¹ and R² have the same meaning as defined above and m′ is an integer comprised between 0 and 7. The preparation of the compound of formula (IIa) from compound of formula (XI) may be carried out under normal condition known by the person skilled in the art, i.e. in the presence of an acid such as Bronsted acid or a Lewis acid. The preparation of the compound of formula (IIa) from compound of formula (XI) may comprise the step of treating compound of formula (XI) with an acid. According to a particular embodiment of the invention, the acid used in the preparation of the compound of formula (IIa) may have a pKa below 3. Specific and non-limiting examples of Bronsted acid may be selected from the group consisting of phosphoric acid, para toluene sulfonic acid, methane sulfonic acid, camphor sulfonic acid, methane disulfonic acid, methane trisulfonic acid, 2,4 dinitrobenzene sulfonic acid. Particularly, the Bronsted acid may be phosphoric acid. Specific and non-limiting examples of Lewis acid may be selected from the group consisting of metal triflates such as Al(OTf)₃, Lanthanide triflates such as Sc(OTf)₃, Bi(OTf)₃, metal tetrafluoroborates such as Zn(BF₄)₂, and metal halide such as AlX₃, RAlX₂, R₂AlX, BX₃ wherein X is an halide and R is a C₁₋₃ alkyl group, ZnCl₂, ZnBr₂.

According to any embodiments of the invention, m′ may be an integer comprised between 0 and 5. Particularly, m′ may be an integer comprised between 0 and 3. Particularly, m′ may be an integer between 0 and 2. Even more particularly, m′ may be 1.

According to any one of the above embodiments of the invention, the compound of formula (XI) is of formula

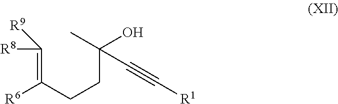

in the form of any one of its stereoisomers or a mixture thereof; and wherein n, R¹, R⁶, R⁸ and R⁹ have the same meaning as defined above.

Non-limiting examples of suitable compounds of formula (XII) may include 3,7-dimethyloct-6-en-1-yn-3-ol, 3,6,7-trimethyloct-6-en-1-yn-3-ol. Compound of formula (IIa) starting from 3,6,7-trimethyloct-6-en-1-yn-3-ol is 1-(3-isopropyl-3-methylcyclopent-1-en-1-yl)ethan-1-one, 1-(3,3,4-trimethylcyclohex-1-en-1-yl)ethan-1-one or a mixture thereof.

The acid can be added into the reaction medium of the invention's process in a large range of concentrations. As non-limiting examples, one can cite as acid concentration values those ranging from 0.01 to 10 equivalents, relative to the amount of the substrate, preferably from 0.1 to 2 equivalent, relative to the amount of substrate. The optimum concentration of the acid will depend, as the person skilled in the art knows, on the nature of the latter, on the nature of the substrate and of the temperature used during the process, as well as the desired time of reaction.

The reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, 1,3-diisopropylbenzene, cumene, pseudocumene, benzyl acetate, xylene or a mixture thereof, C₃₋₁₆ alkane such as hexadecane, hexane, heptane, cyclohexane, ether solvents such as tetrahydrofuran, methyltetrahydrofuran or a mixture thereof. The choice of the solvent is a function of the nature of the substrate and of the nature of the acid and the person skilled in the art is well able to select the solvent most convenient in each case to optimize the reaction.

The temperature at which the preparation of the compound of formula (IIa) from compound of formula (XI) can be carried out is comprised between 50° C. and 180° C. More preferably in the range of between 70° C. and 120° C. Of course, a person skilled in the art is also able to select the preferred temperature as a function of the melting and boiling point of the starting and final products as well as the desired time of reaction or conversion.

According to any embodiments of the invention, the compound of formula (I) being 1-(5,5-dimethylcyclohex-1-en-1-yl)ethan-1-one may further be converted into 1-(5,5-dimethyl-1-cyclohexen-1-yl)-4-penten-1-one. The preparation of 1-(5,5-dimethyl-1-cyclohexen-1-yl)-4-penten-1-one from 1-(5,5-dimethylcyclohex-1-en-1-yl)ethan-1-one is well known in the art such as under allylation conditions. The person skilled in the art will be able to select best conditions to prepare 1-(5,5-dimethyl-1-cyclohexen-1-yl)-4-penten-1-one. So another object of the present invention is a process for the preparation of 1-(5,5-dimethyl-1-cyclohexen-1-yl)-4-penten-1-one comprising the step of
a) isomerizing 1-(3,3-dimethylcyclohex-1-en-1-yl)ethan-1-one by contacting 1-(3,3-dimethylcyclohex-1-en-1-yl)ethan-1-one with a catalyst system comprising
   i) palladium (Pd); and
   ii) molecular hydrogen or a hydrogen source.
   to obtain 1-(5,5-dimethylcyclohex-1-en-1-yl)ethan-1-one
b) converting 1-(5,5-dimethylcyclohex-1-en-1-yl)ethan-1-one into 1-(5,5-dimethyl-1-cyclohexen-1-yl)-4-penten-1-one.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). NMR spectra were acquired using either a Bruker Avance II Ultrashield 400 plus operating at 400 MHz, (¹H) and 100 MHz (¹³C) or a Bruker Avance III 500 operating at 500 MHz (¹H) and 125 MHz (¹³C) or a Bruker Avance III 600 cryoprobe operating at 600 MHz (¹H) and 150 MHz (¹³C). Spectra were internally referenced relative to tetramethyl silane 0.0 ppm. 1H NMR signal shifts are expressed in 8 ppm, coupling constants (J) are expressed in Hz with the following multiplicities: s, singlet; d, doublet; t, triplet; q, quartet; m, multiplet; b, broad (indicating unresolved couplings) and were interpreted using Bruker Topspin software. ¹³C NMR data are expressed in chemical shift δ ppm and hybridization from DEPT 90 and DEPT 135 experiments, C, quaternary(s); CH, methine (d); CH₂, methylene (t); CH₃, methyl (q).

### Example 1

### Preparation of 1-(3,3-dimethylcyclohex-1-en-1-yl)ethan-1-one

500 g (0.6 eq.) of phosphoric acid 85% and 4′400 g Heptane were loaded in the reactor. The mixture was stirred and heated to reflux through dean stark device. 1100 g (1 eq.) of 3,7-dimethyloct-6-en-1-yn-3-ol was fed over 6 h then the reflux was continued for another 4 hours. The reaction mixture was cooled to 60° C. and water was added. The acid phase was discharged and the organic phase was washed with water at 60° C. The solvent was concentrated under vacuum. 1050 g of crude material was obtained resulting to 605 g of 95% pure 1-(3,3-dimethylcyclohex-1-en-1-yl)ethan-1-one after fractional distillation (Bp 80° C./10.9 mbar).

¹H-NMR (500 MHz, CDCl₃), δ (ppm): 6.53 (t, 1H); 2.27 (s, 3H); 2.15 (td, 2H); 1.61 (m, 2H); 1.43 (m, 2H); 1.06 (s, 6H)

¹³C-NMR (125 MHz, CDCl₃), δ (ppm): 199.9; 149.7; 137.3; 36.3; 32.7; 29.1; 25.2; 23.1; 19.1

### Example 2

### Preparation of 1-(3-ethyl-3-methyl-cyclohexen-1-yl)ethan-1-one

Following the procedure in Example 1, 3,7-dimethylnon-6-en-1-yn-3-ol (310.0 g) was converted into 1-(3-ethyl-3-methyl-cyclohexen-1-yl)ethan-1-one (170.5 g, 94% pure) after fractional distillation (Bp 98° C./12.0 mbar).

¹H-NMR (500 MHz, CDCl₃), δ (ppm): 6.55 (t, 1H); 2.28 (s, 3H); 2.12-2.05 (m, 1H); 1.69-1.63 (m, 1H); 1.52-1.33 (m, 4H); 1.02 (s, 3H); 0.87 (t, 3H)

¹³C-NMR (125 MHz, CDCl₃), δ (ppm): 199.8; 149.3; 138.0; 35.7; 34.5; 33.2; 26.1; 25.3; 23.3; 18.9; 8.4

### Example 3

### Preparation of 1-(3-isopropyl-3-methyl-cyclopenten-1-yl)ethan-1-one

Following the procedure in Example 1, 3,6,7-trimethyloct-6-en-1-yn-3-ol (310.0 g) was converted into 1-(3-isopropyl-3-methyl-cyclopenten-1-yl)ethan-1-one (165.8 g, 96% pure) after fractional distillation (Bp 96° C./12.0 mbar).

¹H-NMR (500 MHz, CDCl₃), δ (ppm): 6.52 (t, 1H); 2.55-2.51 (m, 2H); 2.31 (s, 3H); 1.89-1.83 (m, 1H); 1.71-1.65 (m, 1H); 1.59-1.53 (m, 1H); 1.04 (s, 3H); 0.91 (d, 3H); 0.87 (d, 3H)

¹³C-NMR (125 MHz, CDCl₃), δ (ppm): 197.5; 152.2; 143.2; 53.5; 35.9; 33.5; 29.6; 26.7; 22.5; 18.4; 18.0

### Example 4

### Preparation of 1-(3,3-dimethylcyclohexa-1,4-dien-1-yl)ethan-1-one

In an ozonolysis reactor were placed 20.3 g of Δ³-carene, 150 mL MeOH and 150 mL dichloromethane. The resulting solution was cooled down to −78° C. before treatment with O₃ for 2 hours. A solution of triphenylphosphine (40.9 g) in dichloromethane (50 mL) was slowly added over 30 min between −35° C. and −25° C., and the reaction mixture was stirred for 45 min before concentration of the volatiles followed by flash distillation to afford 14.5 g of 95% pure 2-(3-acetonyl-2,2-dimethyl-cyclopropyl) acetaldehyde that was used without further purification.

In a next step, 2-(3-acetonyl-2,2-dimethyl-cyclopropyl) acetaldehyde (20.0 g) was poured into an ice-cold solution of NaOH (18.3 g) in water (170 mL) and EtOH (100 mL). After 16 hours at room temperature, H₂O was added (250 mL) and the mixture was extracted with ethyl acetate (2×100 mL). The combined extracts were then washed with 10% citric acid until pH=5, then with 10% KHCO₃ until pH=8, and finally with H₂O (150 mL). Removal of the solvents from the combined organic phases under reduced pressure followed by fractional distillation (Bp 94° C./11.0 mbar) afforded 14.8 g of 98% pure 1-(3,3-dimethylcyclohexa-1,4-dien-1-yl)ethan-1-one

¹H-NMR (500 MHz, CDCl₃), δ (ppm): 6.62 (q, 1H); 5.69 (dt, 1H); 5.52 (dq, 1H); 2.80 (m, 2H); 2.33 (s, 3H); 1.14 (s, 6H)

¹³C-NMR (125 MHz, CDCl₃), δ (ppm): 199.3; 147.2; 133.2; 121.6; 34.5; 29.9; 27.6; 25.1; 24.4

### Example 5

### Catalytic isomerization of 1-(3,3-dimethylcyclohex-1-en-1-yl)ethan-1-one using Pd/C

Protocol 1:1-(3,3-dimethylcyclohex-1-en-1-yl)ethan-1-one (1430 g, 95%), 28 g of 5% Pd/C (50% water) and 250 g pCymene were stirred at 175° C., then 22.8 g (8% mol) of formic acid were slowly dosed over 20 h. The reaction mixture was cooled to 20° C., filtered off, then subjected to fractional distillation providing 3 fractions being 1-(3,3-dimethylcyclohexyl)ethan-1-one (114 g, Bp 63° C./10 mbar), 1-(3,3-dimethylcyclohex-1-en-1-yl)ethan-1-one (429 g, Bp 78° C./10 mbar) and 1-(5,5-dimethylcyclohex-1-en-1-yl)ethan-1-one (858 g, Bp 83° C./10 mbar).

Protocol 2:1-(3,3-dimethylcyclohex-1-en-1-yl)ethan-1-one (200 g, 95%), and 4 g of 5% Pd/C (50% water) were stirred at 175° C., then a 2% hydrogen solution in nitrogen (corresponding to 8% mol hydrogen) was bubbled through the suspension over 20 h. The reaction mixture was cooled to 20° C., filtered off, then subjected to fractional distillation providing 3 fractions being 1-(3,3-dimethylcyclohexyl)ethan-1-one (14 g, Bp 63° C./10 mbar), 1-(3,3-dimethylcyclohex-1-en-1-yl)ethan-1-one (60 g, Bp 78° C./10 mbar) and 1-(5,5-dimethylcyclohex-1-en-1-yl)ethan-1-one (120 g, Bp 83° C./10 mbar).

¹H-NMR (700 MHz, CDCl₃), δ (ppm): 6.87 (m, 1H); 2.28 (m, 2H); 2.28 (s, 3H); 2.01 (q, 2H); 1.34 (t, 2H); 0.9 (s, 6H)

¹³C-NMR (175 MHz, CDCl₃), δ (ppm): 199.5; 139.7; 138.7; 36.4; 34.2; 28.5; 28.0; 25.3; 24.1

### Example 6

### Catalytic isomerization of 1-(3-ethyl-3-methyl-cyclohexen-1-yl)ethan-1-one using Pd/C

Catalytic isomerization of 1-(3-ethyl-3-methyl-cyclohexen-1-yl)ethan-1-one was performed following Protocol 1 described in Example 5. The final composition was determined by GC-MS and NMR analyses, and a conversion of 69% was obtained. Purification of the crude reaction mixture on column chromatography afforded 49% of 1-(5-ethyl-5-methyl-cyclohexen-1-yl)ethan-1-one. No other regioisomer was detected.

¹H-NMR (500 MHz, CDCl₃), δ (ppm): 6.89 (m, 1H); 2.29 (s, 3H); 2.28-2.25 (m, 2H); 2.01 (td, 2H); 1.37-1.35 (m, 2H); 1.29-1.24 (m, 2H); 0.85 (t, 3H); 0.83 (s, 3H)

¹³C-NMR (125 MHz, CDCl₃), δ (ppm): 199.6; 140.1; 138.6; 34.8; 33.5; 31.9; 31.1; 25.4; 23.9; 23.7; 7.8

### Example 7

### Catalytic isomerization of 1-(3,3-dimethylcyclohexa-1,4-dien-1-yl)ethan-1-one using Pd/C

1-(3,3-dimethylcyclohexa-1,4-dien-1-yl)ethan-1-one was isomerized following Protocol 2 described in Example 5. Formation of 1-(5,5-dimethylcyclohexa-1,3-dien-1-yl)ethan-1-one was determined by GC-MS and NMR analyses, and a conversion of 55% was obtained for a yield of 15%.

¹H-NMR (500 MHz, CDCl₃), δ (ppm): 6.88 (d, 1H); 6.01-5.95 (m, 2H); 2.36 (s, 2H); 2.33 (s, 3H); 1.01 (s, 6H)

¹³C-NMR (125 MHz, CDCl₃), δ (ppm): 199.2; 153.2; 136.1; 132.7; 117.5; 41.2; 34.0; 27.7; 27.1

### Example 8

### Catalytic isomerization of 1-(3-isopropyl-3-methyl-cyclopenten-1-yl)ethan-1-one using Pd/C

1-(3-isopropyl-3-methyl-cyclopenten-1-yl)ethan-1-one was isomerized following Protocol 2 described in Example 5. Formation of 1-(4-isopropyl-4-methyl-cyclopenten-1-yl)ethan-1-one was determined by GC-MS and NMR analyses, and a conversion of 63% was obtained. No other regioisomer was detected.

### Example 9

### Catalytic isomerization of 1-(3,3-dimethylcyclohex-1-en-1-yl)ethan-1-one using Pd/C in EtOH (Comparative Example)

Isomerization of 1-(3,3-dimethylcyclohex-1-en-1-yl)ethan-1-one was performed under the same reaction conditions than described by Takasago (EP 1 162 190 A2), replacing RhCl₃·3H₂O catalyst by Pd/C. No isomerization occurred after 23 h.

1. A process for preparing a 1-(cycloalk-1-en-1-yl) acyl or 1-(cycloalkadien-1-yl) acyl derivative, the process comprising isomerizing intracyclic carbon-carbon double bond(s), wherein said isomerizing is carried out by contacting a substrate comprising a conjugated intracyclic carbon-carbon double bond with a catalyst system comprising
i) palladium (Pd); and
ii) molecular hydrogen or a hydrogen source.

2. The process according to claim 1, wherein the 1-(cycloalk-1-en-1-yl) acyl or 1-(cycloalkadien-1-yl) acyl derivative is a compound of formula 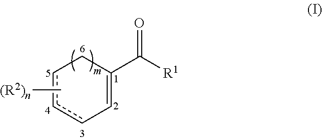
in a form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents a substituent of the ring and is a C₁₋₆ alkyl or C₂₋₆ alkenyl group; or two R² groups or R¹ and one R² group, taken together, form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

3. The process according to claim 1, wherein the substrate is a compound of formula 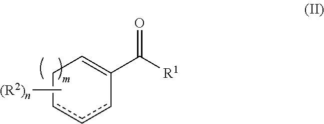
in a form of any one of its stereoisomers or a mixture thereof and wherein one dotted line is a carbon-carbon single bond and the other is a carbon-carbon single bond or a carbon-carbon double bond; m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents a substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

4. The process according to claim 2, wherein the compound of formula (I) is of formula 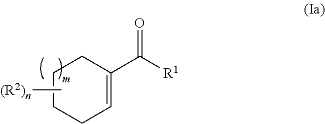
in a form of any one of its stereoisomers or a mixture thereof and wherein m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents a substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group; and
the substrate is a compound of formula 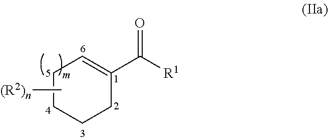
in a form of any one of its stereoisomers or a mixture thereof and wherein m is an integer comprised between 0 and 7; n is a integer comprised between 1 and 8; R¹ is a C₁₋₆ alkyl, C₂₋₆ alkenyl or C₁₋₆ alkoxy group; and each R², simultaneously or independently, represents a substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

5. The process according to claim 4, wherein m is an integer comprised between 0 and 2.

6. The process according to claim 4, wherein each R², simultaneously or independently, is a C₁₋₃ alkyl group; or two R² groups, taken together form a C₅₋₆ cycloalkyl group.

7. The process according to claim 4, wherein the compound of formula (I) is a compound of formula 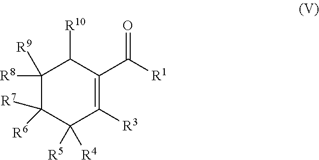
in a form of any one of its stereoisomers or as a mixture thereof, wherein R¹ is a C₁₋₆ alkyl, C₂₋₆ alkenyl or C₁₋₆ alkoxy group; each R³, R⁴, R⁵, R⁶, R⁷, R⁸, R⁹ and R¹⁰, independently from each other, represent a hydrogen atom, a C₁₋₆ alkyl or C₂₋₆ alkenyl group; or two groups among R³, R⁴, R⁵, R⁶, R⁷, R⁸, R⁹ and R¹⁰ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R³ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R⁴ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; provided that R³, R⁴, R⁵, R⁶, R⁷, R⁸, R⁹ and R¹⁰ are such that compound of formula (V) is different from a compound of formula (VI), wherein;
the compound of formula (II) is of formula 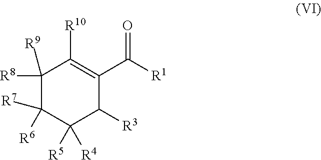
in a form of any one of its stereoisomers or as a mixture thereof, wherein R¹ is a C₁₋₆ alkyl, C₂₋₆ alkenyl or C₁₋₆ alkoxy group; each R³, R⁴, R⁵, R⁶, R⁷, R⁸, R⁹ and R¹⁰ independently from each other, represent a hydrogen atom, a C₁₋₆ alkyl or C₂₋₆ alkenyl group; or two groups among R³, R⁴, R⁵, R⁶, R⁷, R⁸, R⁹ and R¹⁰ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R³ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group; or R⁴ and R¹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

8. The process according to claim 7, wherein the compound of formula (V) is a compound of formula 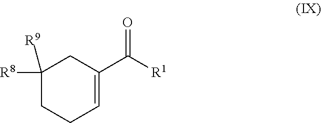
in a form of any one of its stereoisomers or as a mixture thereof, wherein R¹ is a C₁₋₆ alkyl, C₂₋₆ alkenyl or C₁₋₆ alkoxy group; each R⁸ and R⁹, independently from each other, represent a hydrogen atom, a C₁₋₆ alkyl or C₂₋₆ alkenyl group; or R⁸ and R⁹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group;
and the compound of formula (VI) is of formula 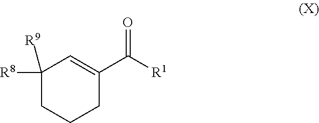
in a form of any one of its stereoisomers or as a mixture thereof, wherein R¹ is a C₁₋₆ alkyl, C₂₋₆ alkenyl or C₁₋₆ alkoxy group; each R⁸ and R⁹, independently from each other, represent a hydrogen atom, a C₁₋₆ alkyl or C₂₋₆ alkenyl group; or R⁸ and R⁹ are taken together and form a C₅₋₈ cycloalkyl or C₅₋₈ cycloalkenyl group, each optionally substituted by one or more C₁₋₆ alkyl, C₂₋₆ alkenyl or C₁₋₆ alkoxy group.

9. The process according to claim 4, wherein R¹ is a C₁₋₃ alkyl group and n is 2.

10. The process according to claim 1, wherein the palladium is supported on carbon.

11. The process according to claim 1, wherein said hydrogen source is tetralin, formic acid, formate salt, limonene or a mixture thereof.

12. The process according to claim 1, wherein said process is a continuous process.

13. The process according to claim 4, wherein the process further comprises preparing the compound of formula (IIa) from a compound of formula 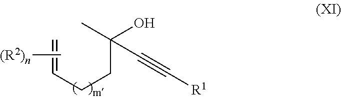
in a form of any one of its stereoisomers or a mixture thereof; wherein n is a integer comprised between 1 and 8; R¹ is a C₁₋₆ alkyl, C₂₋₆ alkenyl or C₁₋₆ alkoxy group; and each R², simultaneously or independently, represents a substituent of the ring and is a C₁₋₆ alkyl or C₂₋₆ alkenyl group; or two R² groups or R¹ and one R² group, taken together form a C₅₋₈ cycloalkyl or C₅₋₈ cycloalkenyl group, each optionally substituted by one or more C₁₋₆ alkyl, C₂₋₆ alkenyl or C₁₋₆ alkoxy group and m′ is an integer comprised between 0 and 7.

14. The process according to claim 13, wherein the preparing the compound of formula (IIa) from the compound of formula (XI) comprises a step of treating the compound of formula (XI) with an acid.

15. A process for preparing 1-(5,5-dimethyl-1-cyclohexen-1-yl)-4-penten-1-one comprising
a) isomerizing 1-(3,3-dimethylcyclohex-1-en-1-yl)ethan-1-one by contacting 1-(3,3-dimethylcyclohex-1-en-1-yl)ethan-1-one with a catalyst system comprising
i) palladium (Pd); and
ii) molecular hydrogen or a hydrogen source,
to obtain 1-(5,5-dimethylcyclohex-1-en-1-yl)ethan-1-one; and
b) converting 1-(5,5-dimethylcyclohex-1-en-1-yl)ethan-1-one into 1-(5,5-dimethyl-1-cyclohexen-1-yl)-4-penten-1-one.

16. The process according to claim 4, wherein m is 1.

17. The process according to claim 4, wherein each R², simultaneously or independently, is a methyl group.
